(19) United States
(12) Patent Application Publication
(10) Pub. No.: US 2004/0236269 A1
(43) Pub. Date: Nov. 25, 2004

(54) Microsurgical tissue treatment system

(76) Inventors: Kevin S. Marchitto, Golden, CO (US); Stephen T. Flock, Arvada, CO (US)

Correspondence Address: Benjamin Aaron Adler, ADLER & ASSOCIATES, 8011 Candle Lane, Houston, TX, 77071, US

(21) Appl. No.: 10/808,108

(22) Filed: Mar. 24, 2004

Related U.S. Application Data
(63) Continuation-in-part of application No. 10/670,618, filed on Sep. 25, 2003.
(60) Provisional application No. 60/413,351, filed on Sep. 25, 2002.

(51) Int. Cl. A61B 17/20

(52) U.S. Cl. 604/22

(57) ABSTRACT

Provided herein are devices and methods for altering a tissue in an individual. The device comprises an applicator having a surface to contact the tissue, a means to drive the applicator and, optionally, an abrasive material. The devices can further comprise a means to monitor a physical property, such as an electrical property, of the tissue with feedback control. The devices and methods may be used to partially or completely ablate the skin or other tissue in an individual for the purpose of enhancing the delivery of a therapeutic or diagnostic material or molecule or to improve cosmesis.

### CROSS-REFERENCE TO RELATED APPLICATIONS

This application is a continuation-in-part of patent application U.S. Ser. No. 10/670,618, filed Sep. 25, 2003, which claims benefit of provisional U.S. Ser. No. 60/413,351, filed Sep. 25, 2002, now abandoned.

### BACKGROUND OF THE INVENTION

1. Field of the Invention

The present invention relates generally to the fields of biomedical engineering and drug delivery and tissue microsurgery. More specifically, the present invention provides a device and methods for treating tissues, for controlling the permeation rates of substances across biological membranes and for achieving consistent results between treatment sites, as well as for tissue treatment for therapeutic or cosmetic reasons.

2. Description of the Related Art

Various methods have been used for facilitating the delivery of compounds across the skin and other membranes. In general, permeation of drugs through the skin occurs at a very slow rate, if at all. The primary rate-limiting step in this process is the passage of these compounds through the outermost layer of skin, called the stratum corneum. The stratum corneum is a very thin layer of dead cells that acts as an impermeable layer to matter on either side of this layer. The stratum corneum primarily provides the skin's barrier function. It has long been recognized that loss or alteration of the stratum corneum results in increased permeability to many substances; materials can more easily diffuse into or out of the skin. Several approaches have been used to ablate the stratum corneum for the purposes of drug delivery, however most fail to address the need for a high degree of precision and accuracy for consistent alterations of the stratum corneum and drug flux through the skin.

U.S. Pat. No. 6,424,863, U.S. Pat. No. 6,425,873, U.S. Pat. No. 6,315,722, U.S. Pat. No. 6,251,100, U.S. Pat. No. 6,056,738 and U.S. Pat. No. 5,643,252 demonstrate that electromagnetic energy induced alterations of the stratum corneum result in increased permeability to substances. Alternatively, compounds referred to as permeation enhancers, e.g., alcohol or drug carriers such as liposomes, can be used with some success to penetrate the stratum corneum. The barrier function of the skin presents a very significant problem to pharmaceutical manufacturers interested in topical administration of drugs, or in transcutaneous collection of bodily fluids.

U.S. Pat. No. 4,775,361 provides that electromagnetic energy produced by lasers may be used to ablate the stratum corneum in order to make the skin more permeable to pharmaceutical substances. Devices and methods for drug delivery using laser ablation systems have been described. U.S. Pat. No. 6,251,100 provides an improved method of administering a pharmaceutical composition, such as an anesthetic, through the skin of a patient without the use of a sharp or needle. This method includes the step of irradiating the stratum corneum of a region of the skin of the patient using a laser. By a selection of parameters, the laser irradiates the surface of the skin precisely to a selectable depth, without causing clinically relevant damage to healthy proximal tissue. A pharmaceutical composition is then applied to the region of irradiation. International Publication WO 00/57951 describes the use of non-ionizing energy, including lasers, to improve methods of administering pharmaceuticals in tissues, including the skin.

Devices and methods in U.S. Pat. No. 5,683,366, U.S. Pat. No. 5,697,536, U.S. Pat. No. 6,228,078, and U.S. Pat. No. 5,888,198 describe bipolar and monopolar RF electrosurgical devices that use a method of tissue disintegration as a means to ablate tissue prior to myocardial revascularization, tissue resurfacing or other surgical procedures. Radiofrequency energy has also been used to ablate tissues, and related methods have been used to enhance drug delivery through the skin. Publication WO 00/57951 describes methods and devices that use radiofrequency energy to improve permeation of substances across the stratum corneum. Certain applications describe feedback mechanisms that are used to prevent damage to viable tissue in the area surrounding the treatment site, such as described in U.S. Patent Publication No. 2002/0010414 A1 and WO 01/21068.

There are many procedures where a very precise ablation of tissue is of therapeutic benefit. For example, in ossicular bone surgery, the bones of the middle ear are sometimes removed, reshaped with a high-speed drill, and then reinserted in order to treat middle ear disease; the success of this procedure is very dependent upon surgical skill as the drill can remove large amounts of bone, if not used properly. In corneal sculpting or keratomileusis, tissue in the eye is reshaped with a laser in order to correct for vision disorders, such as myopia. These lasers are very expensive and require significant safety mechanisms because they employ potentially hazardous radiant energy. Dentists use several different tools to debride teeth of bacterial plaque and calculus, polish teeth and reshape teeth for aesthetic purposes. These tools can be inappropriate for very delicate and precise procedures and the result depends greatly on the skill of the dentist or oral hygienist.

A multitude of ablative procedures are performed on the visible surfaces of various tissues in order to improve their appearance, e.g., as in cosmetic tissue resurfacing treatments. Microdermabrasion is a common procedure where a thin layer of skin is removed with chemicals or a high-speed jet of crystals, whereupon small wrinkles or faults are smoothed out, as well as irregularities due to photodamage, acne scarring and scarring from surgical trauma. This process improves the appearance of the skin by giving it a smooth, fresh look. Conventional dermabrasion uses either a diamond fraise or a wire brush as a cutting tool powered by a handheld high-speed motor. The disadvantages of the powered tool include aerosolizing of infectious particles and blood splatter. Others have reported back-and-forth or circular motion manual use of abrading devices, including wire brushes or sandpaper.

A similar ablative process is also done, sometimes with lasers, for the purposes of burn debridement or scar revisions. Nail shaping and polishing also use an ablative process, although it is usually done manually by a trained person. Hair removal can be done several different ways, but the most popular for large areas of hair involve a lasers which ablates skin and sensitive parts of the hair follicle, however, such lasers are extremely expensive and require extensive training of the provider.

Recently, U.S. patent publication 200258902 described methods and devices for the ablation of barrier membranes using a shear device in order to enable sampling of biological fluids for diagnostic purposes and to enable delivering of active compounds for therapeutic purposes. That invention features a method for transporting a molecule through a mammalian barrier membrane following the ablation of the membrane with a shear device comprising a shear sheet containing at least one opening and a shear member, e.g., a shear blade such as those used in electric razors, where the sheet is contacted with the membrane such that a portion of the membrane is forced through the opening and the shear member as it moves parallel to the shear sheet, ablates the portion of the membrane exposed through the opening; the shearing process must be followed by a driving force to move the relevant molecule through the sheared membrane. The device further comprises a sensor, the feedback from which that modifies the driving force, e.g., by starting, speeding up, slowing down, or stopping the shear member's motion to enhance sufficient but not excessive membrane ablation.

Recently, methods and devices for transiently reducing the barrier function, i.e. increasing the permeation of topically applied drugs, have been described whereby the operative physical event involves ultrasonic waves. For example, U.S. Pat. Nos. 6,620,123, 6,002,961 and 6,190,315 describe the use of low-frequency ultrasonic waves, i.e. 20,000 Hz, delivered to the skin surface with a transducer coupled to the tissue by a conductive medium. When used to treat tissue, this approach renders the barrier function transiently reduced. The causative physical event is purportedly the production of cavitation bubbles within the stratum corneum.

For both drug delivery and biological fluid sampling, non-invasive and minimally invasive methods are preferred over invasive methods, such as needle injection, since they may easily be self-administered and are pain free. U.S. Pat. Nos. 5,250,028 and 5,843,113, PCT Patent Applications Nos. WO98/11937 and WO97/48440, and Henry et al. (1), disclose perforation or disruption of the skin barrier membrane with mechanical means, e.g., with either small blades or needles, for such purposes. U.S. Pat. Nos. 5,421,816; 5,445,611 and 5,458,140 disclose, as a replacement for invasive sampling, the use of ultrasound to act as a pump for expressing interstitial fluid directly through visually intact, i.e., non-lanced, skin. Other means of treating a tissue to transiently increase the tissue permeability to enhance molecular transport for drug delivery and/or for sampling of interstitial fluids are disclosed in U.S. Pat. Nos. 5,019,034, 5,547,467, 5,667,491, 5,749,847, 5,885,211, and 5,441,490 and PCT Patent Application WO 95/12357.

It is notable that a consistent means of treatment are desirable. The Code of Federal Regulations (21 CFR 860.7(e)(1)) establishes that there is “reasonable assurance that a device is effective when it can be determined, based upon valid scientific evidence, that in a significant portion of the target population, the use of the device . . . will provide clinically significant results.” Devices which cannot be shown to provide consistent results between patients, or even within a single patient upon multiple use, will have minimal utility and may not be approvable for broad use.

Beyond devices it is generally desirable to develop medical products with critical controls that can deliver a precise result. Of critical concern is the delivery of many drugs. Certain drugs can be described as having a “broad” or “narrow” therapeutic index (TI). That is some drugs may be useful over a broad range of concentrations and thus are safe for the general population, while other drugs may only be effective over a narrow concentration range and may even be dangerous when administered in greater than recommended concentrations. This is particularly true where a drug has a narrow TI; the delivery of the drug must be controlled carefully so as to avoid potential harmful effects. The FDA (PMA Memorandum #P91-1: Clinical Utility and Pre-market Approval) has established that devices which cannot be controlled may have limited utility. In particular, a drug delivery device may have limited utility if no assurance can be made that a consistent dosage is delivered throughout the patient population.

The drug-device combination must be capable of consistently delivering a dosage. As part of INDs and NDAs for administered drug products, bioavailability studies focus on determining the process by which a drug is released from the administered dosage form and moves to the site of action. Bioavailability data provide an estimate of the fraction of the drug absorbed, as well as its subsequent distribution and elimination. Bioavailability is defined in 21 CFR 320.1 as “the rate and extent to which the active ingredient or active moiety is absorbed from a drug product and becomes available at the site of action. For drug products that are not intended to be absorbed into the bloodstream, bioavailability may be assessed by measurements intended to reflect the rate and extent to which the active ingredient or active moiety becomes available at the site of action.” This definition focuses on the processes by which the active ingredients or moieties are released from a dosage form and move to the site of action. A delivery device which does not consistently release the same levels of a drug product due to the design of a product will have limited clinical utility, as there can be no assurance that a certain dosage has been delivered at any point in time.

Furthermore, studies to establish bioequivalence between two products are important to demonstrate safety and therapeutic efficacy in a product, and will be a benchmark for approval of drugs by regulatory bodies. Bioequivalence is defined at 21 CFR 320.1 as “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study.”

As noted in the statutory definitions, both bioequivalence and product quality bioavailability focus on the release of a drug substance from a drug product and subsequent absorption into the systemic circulation. Where the test product generates variable effect at the site of action, as compared to those of the reference product, the product cannot be claimed as consistent, will not have great clinical utility and could be dangerous to use.

Control of delivery for current “patch” transdermal applications is achieved by delivering a fraction of what is “absorbable,” and either regulating the size of the dosage or the amount which is released from the vehicle. However, this simple means of regulation is not adequate for a system that could greatly accelerate the rate of percutaneous absorption. The condition of the skin and its hydration are significant factors in the percutaneous absorption of drugs. Some solubility of the substance in both lipid and water is thought to be essential. The aqueous solubility of a drug determines the concentration presented to the absorption site and the partition coefficient strongly influences the rate of absorption across the absorption site (Pharmaceutical Dosage Forms and Drug Delivery Systems, Ansel, H. C., Popovich, N. G. Allen, L. V. Eds., Williams & Wilkins, Baltimore, 1995). Vehicles that increase the hydration of the skin generally favor percutaneous absorption of drugs.

Whereas mechanisms are known in the art for protecting viable tissue surrounding the treatment site, the prior art is deficient in methods to achieve control over the alteration event in order to achieve variable rates of permeability. It would be beneficial for delivery devices to deliver consistently reliable dosages between sites and across a patient population or to assure that a consistent amount of material is collected from a site by adjusting the permeability characteristics of the treatment site itself, in addition to traditional methods in the formulation. Another benefit is recognized in the ability to regulate the depth of treatment as it relates to possible toxicity as well as flux, i.e., rate of permeation, and the surface area of the treatment site with relation to flux. Furthermore, it is desirable to attain simultaneous delivery of substances with minimal generation of heat.

Current cosmetic and therapeutic tissue treatments either involve a very expensive instrument, such as a laser, and highly trained care provider or the quality of the result depends greatly on the person doing the treatment. The inventors have recognized a need in the art for a device and methods that can alter tissue to produce a precise treatment with a high degree of control and that can be done economically and optionally at home by the untrained individual desirous of the treatment. The present invention further fulfills this long-standing need and desire in the art.

### SUMMARY OF THE INVENTION

The present invention is directed to device for treating a tissue in an individual. The device comprises an applicator having a surface disposed to contact the tissue and a means to drive said applicator. The surface may comprise an abrasive and/or a pharmaceutical and/or a lubricant. The device further may comprise a means to dispense a therapeutic or diagnostic material and/or a means to collect ablated tissue or a biomolecule after treatment. Also the device may comprise a means to monitor an electrical property of the tissue.

The present invention also is directed to a method for treating tissue. The tissue is contacted at a site of interest with the surface of the applicator of the device described herein and the applicator is actuated via a driving means. The tissue is altered at the site of interest thereby treating it. Additionally, the method may comprise dispensing a pharmaceutical to the altered tissue. The method also may comprise monitoring an electrical property of the tissue at the site of alteration. An electric current generated between active and return electrodes at the site of interest or eddy currents at the site of interest generated via an alternating magnetic field may be monitored to provide information about conductivity, impedance or hydration of the altered tissue.

The present invention is directed further to a method of controlling the permeability of a tissue in an individual. The tissue is contacted at a site of interest with the surface of the applicator of the device described herein and altered via actuation of the device and an electrical property of the tissue at the site of interest is monitored. An algorithm is applied to evaluate the electrical property and the value obtained for the electrical property is compared to a predetermined value whereby the values correlate to the permeability of the tissue. If the obtained value does not at least equal said predetermined value the device is signaled via said controller to continue altering the tissue thereby controlling the permeability of the tissue at the site of interest. The method further may comprise dispensing a pharmaceutical to the site of interest during the monitoring step or subsequent to reaching the predetermined value of the electrical property.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIG. 1 depicts an actuator delivery system having an actuator encased in a housing to form a vibrating probe.

FIG. 2 depicts a cross-sectional view of the device of FIG. 1 when used on skin.

FIG. 3 depicts the surface of the actuator that is placed against the membrane to be treated displaying an array of chevrons on the inferior surface.

FIG. 4 depicts another embodiment of FIG. 1 where the probe and piezoelectric actuator are associated with at least one electrode that is in electrical contact with the ablation site of the membrane.

FIG. 5 depicts a horizontal displacement actuator.

FIG. 6 depicts a device that ablates the tissue or transiently disrupts the barrier properties of the tissue.

FIGS. 7A-7B depict two different applicators which, when in contact with tissue, ablates or transiently disrupts the barrier properties of the tissue.

FIG. 8 depicts another device which, when in contact with tissue, serves to ablates or transiently disrupts the barrier properties of the tissue.

FIG. 9A depicts a treatment device that can be stacked to produce motion in at least two dimensions. FIG. 9B shows a device that causes a mechanical deformation in a circular pattern.

FIGS. 10A and 10B depict a device consisting of coils, or other energy applicators, which are used to move an element in a way suitable to treat skin.

### DETAILED DESCRIPTION OF THE INVENTION

A device for treating a tissue in an individual, comprising an applicator having a surface disposed to contact the tissue; and a means to drive said applicator. Further to this embodiment the device comprises a means to dispense a therapeutic or diagnostic material. Also, the device comprises a means to collect ablated tissue or a biomolecule after treating the tissue. Again further to this embodiment the device may comprise a means to house the device, optionally having two wheels rotably attached thereto. Furthermore, the device may comprise a means to monitor an electrical property of the tissue.

In aspects of this embodiment the means to drive the applicator may be a single stage actuator or a multi-stage actuator. The driving means may be a piezoelectric actuator, an electrorestrictive actuator, a magnetorestrictive actuator, or a high frequency electronic motor or a combination thereof. Additionally, the driving means may be magnetically responsive.

In other aspects of this embodiment, the applicator produces vibratory motion at a frequency from about 1 Hz to about 40,000 Hz. The surface of the applicator may be textured. The surface of the applicator may comprise an abrasive material which may have a particle size of about 30 microns to about 120 microns. In these aspects the abrasive material may be diamond, aluminum oxide or carborundum. The abrasive material may be crystalline. An example of a crystalline abrasive is ice. The abrasive may comprise a pharmaceutical, a biologic or a diagnostic. The surface of the applicator may comprise a lubricant with or without the abrasive. Examples of a lubricant are water, a hydrogel, a lipid, aqueous carbohydrate, petrolatum, an inorganic oil, glycerol or a combination thereof.

In other aspects of this embodiment the dispensing means may comprise a reservoir containing the therapeutic or diagnostic material and a permeable membrane through which the therapeutic or diagnostic material is controllably released. Alternatively, the dispensing means may be the abrasive which is or comprises the therapeutic or diagnostic material. Optionally, the abrasive may comprise a lubricant.

In this aspect the therapeutic or diagnostic material may be a pharmaceutical, an anesthetic, nitroglycerin, an anti-nauseant, an antibiotic, a hormone, a steroidal anti-inflammatory agent, a non-steroid anti-inflammatory agent, a chemotherapeutic agent, an anti-cancer agent, an immunogen, an anti-viral agent or an anti-fungal agent, or a diagnostic material. Representative examples of these types of pharmaceuticals are as disclosed infra.

In additional aspects of this embodiment the collection means may be a container operably connected to the device. Alternatively, the collection means may be an absorptive medium. Examples of an absorptive medium are activated carbon, a dehydrated hydrogel or cotton.

In yet another aspect of this embodiment the monitoring means comprises at least one active electrode to electrically contact the tissue at a site of interest; a second return electrode distal to the first electrode to electrically contact the tissue at the site of interest; an optional electrically conductive fluid interface between the first and second electrodes and the site of interest on the tissue; and a controller to monitor an electrical current between the first electrode(s) and the second electrode. Alternatively, the monitoring means comprises a source of an alternating magnetic field to generate eddy currents in the tissue; a detector to detect the eddy currents. In both aspects the controller further comprises a microprocessor. Examples of the electrical property are conductivity, impedance or hydration.

In another embodiment of the present invention there is provided a method for treating tissue, comprising contacting the tissue at a site of interest with the surface of the applicator of the device described supra; actuating the applicator comprising the device via the driving means; and altering said tissue at the site of interest thereby treating the tissue. Further to this embodiment the method comprises dispensing a pharmaceutical, a biologic or a diagnostic to said altered tissue. Additionally, the method comprises monitoring an electrical property of the tissue to control the alteration thereof.

In one aspect of this embodiment the dispensing means dispenses the pharmaceutical, biologic or diagnostic simultaneously with the tissue alteration or subsequently to the tissue alteration. The pharmaceutical, biologic or diagnostic are as described infra.

In another aspect of this embodiment monitoring the electrical property of the tissue comprises contacting the tissue at the site of interest with at least one active electrode and a return electrode distal to the electrode; monitoring an electrical current between the active electrode(s) and the return electrode via a controller; and

correlating said electric current with the electrical property of the tissue. Further to this aspect the method may comprise applying an electrically conductive fluid interface between the active electrode(s) and the return electrode and the site of interest on the tissue. The electrical property may be conductivity, impedance or hydration.

In an alternative aspect the monitoring the electrical property comprises providing an alternating magnetic field proximally to the tissue; generating eddy currents via the magnetic field in the tissue; monitoring the eddy currents via a controller; and correlating the eddy currents with electrical conductivity of the tissue. In both aspects of monitoring an electrical property the method further may comprise signaling the device described supra to continue altering the tissue or to adjust the altering of the tissue at the site of interest via a microprocessor in the controller.

In all aspects of this embodiment the tissue may be membraneous or non-membraneous. An example of a membraneous tissue is the stratum corneum. An example of a non-membraneous tissue is bone. In all aspects treating the tissue comprises at least partial ablation of the tissue, a reduction of the mechanical integrity of the tissue, dermabrasion, or a cosmetic procedure.

In yet another embodiment of the present invention there is provided a method of controlling the permeability of a tissue in an individual comprising contacting the tissue at a site of interest with the surface of the applicator of the device described supra; altering the tissue at the site of interest; monitoring an electrical property of the tissue at the site of interest; applying an algorithm to evaluate the electrical property; comparing the value obtained for the electrical property to a predetermined value wherein the values correlate to the permeability of the tissdetermining if the obtained value is at least equal to the predetermined value; and signaling the device via the controller to continue altering the tissue if the obtained value does not at least equal the predetermined value thereby controlling the permeability of the tissue at the site of interest. Further to this embodiment the method comprises dispensing a pharmaceutical to the site of interest, where the pharmaceutical is dispensed during the monitoring step or subsequent to reaching the predetermined value of the electrical property. The pharmaceuticals are described supra.

In an aspect of this embodiment monitoring the electrical property of the tissue comprises contacting the tissue at the site of interest with at least one active electrode and a return electrode distal to the electrode and monitoring an electrical current between the active electrode(s) and the return electrode via a controller. Furthermore, the method may comprise applying an electrically conductive fluid as described supra. Alternatively, monitoring the electrical property comprises providing an alternating magnetic field proximally to the tissue; generating eddy currents in the tissue via the magnetic field; and monitoring the eddy currents via a controller.

In all aspects of this embodiment the electrical property may be conductivity, impedance or hydration. The predetermined value of the electrical property is a known value or is obtained prior to treating the tissue. Furthermore, the predetermined value of the electrical property is obtained from the same individual or within a group of individuals.

The present invention provides a device for removal of thin layers of tissue and methods of use. Generally, the device comprises an abrasive member or applicator and a high frequency drive mechanism. The drive mechanism preferably is, but not limited to, a piezoelectric actuator. The actuator causes high frequency vibration in the plane defined by the tissue surface in at least one dimension relative to the site of treatment. Optionally, simultaneous motion in two or even three dimensions may be beneficial. Additional drive mechanisms may be coupled to the device to achieve motion in additional dimensions.

The present invention further provides methods of treating tissue to remove or alter the tissue in a controlled manner, without generating deleterious heat. Highly controlled treatment results in consistency across the specimen. When ablating tissue, for example, an object of the invention is to remove tissue consistently and evenly at the interface of the applicator. Typically, smooth polished surfaces may be obtained, particularly for cosmetic applications. Thus, use of the device in the methods disclosed herein result in removal of excess tissue, cosmetic resurfacing and other cosmetic applications, including dermabrasion for smoothing of the skin and for reduction of wrinkles. These effects can be obtained by treating surfaces at relatively high frequencies.

Alternatively, the tissue alteration may involve disruption of the barrier function of the tissue, e.g. stratum corneum, with vibrational energy arising from mechanical events, such as mechanical reduction, without actual tissue ablation or with partial ablation. The disruption of the barrier function may be transient.

The means to drive the applicator may incorporate components capable of operating at high frequencies, such as a piezoelectric, an electro- or magnetorestrictive element, an electrical motor, an electrical vibrator motor, or combinations thereof. Furthermore, the drive mechanism may also include solenoids, high-pressure gas, explosive material, voice-coil, electro- or magneto-responsive materials, e.g., polypyrrol or magnetorestrictive elements, electro- or magneto-rheologic materials, e.g., metallic or magnetic filings dispersed in a viscous fluid, shape-memory alloys or polymers, e.g., Nitinol. These driving means may be used singly or in combination.

The device may use an additional driving force to permeate substances into a site treated by the abrasive member. The driving force may optionally include, but not be limited to, electrophoretic means, mechanical pressure, diffusion gradients, osmotic pressure or hydrostatic pressure. Additionally, asymmetric weights and/or textured abraders may be attached to the motors in order to assist the ablation or alteration of tissue with which they come in contact.

Where sufficient force is not available from a single driving element, i.e., a single-stage actuator, a second or third driving element operating in another dimension could provide appropriate force for the actuator, i.e., a multi-stage actuator. For example, some piezoelectric materials operate at sufficiently high frequencies, however, they do not generate enough force to shear tissue, particularly when driven by low power. Mounting such a device on a second actuator, such as an electric motor, in a second dimension, may provide appropriate force while still taking advantage of the high frequency lubricating effect of the piezoelectric transducer. High frequency motion may be obtained by other means as well. For example, exposing magnetic-responsive materials to alternating magnetic fields at high frequency can result in a high frequency vibration effect.

The applicator, alone or in combination, may be driven at high frequencies, for example, between 1 KHz and 40 MHz. These frequencies provide a substantial lubricating effect so tissue is controllably removed and not torn, ripped or shredded. The applicator may be driven by materials operating at high frequencies provided they generate sufficient force to cause an effect.

The applicator further may comprise a surface that is disposed adjacent or in contact with the tissue to be treated. The surface may be textured, may comprise an abrasive material or a lubricant or any combination thereof. Furthermore, the applicator may comprise a means to dispense or to deliver a therapeutic or diagnostic material. For example, the therapeutic or diagnostic material may be contained within a reservoir which has a permeable membrane to control release. Alternatively, the therapeutic or diagnostic material may be or comprise the abrasive and an optional lubricant.

A safety interlock may be affixed to the device, or integrated into a patch, such that the device cannot be utilized unless the interlock is engaged, and only under proper use. For example, the interlock could be mechanical, electrical or optical. In the “on” position, either engaged or disengaged, the device may be operational. In the “off” position, the device would fail to be operational. This interlock prevents treating beyond a subscribed depth, and also prevents subsequent use of the same abrasive material on another patient.

A container may be attached to the distal end of the device such as to contain the abrasive and collect ablated tissue or other biomolecule. The container may be permanent or disposable. Alternatively, in a patch device, the container would be equivalent to a disposable or non-disposable component that is in contact with the skin. The container may be modified to hold, or receive through an opening, a pharmaceutical or other substance, which may then be delivered or collected simultaneously or shortly after ablating of the tissue occurs. The container may be integral to, or function independently of a safety interlock. Alternatively, an absorptive material may be used, e.g., activated carbon, dehydrated hydrogel or cotton.

A control means to monitor feedback about a physical property of the tissue may be used. The control means comprises monitoring various physical properties of tissue such as properties that can change when tissue is ablated or altered. For example, electrical impedance, electrical capacitance, pH, optical fluorescence or reflectance, either IR or visible, thermal diffusivity and conductivity, transepithelial water loss, ultrasonic reflection, or gaseous efflux may be measured.

In the case of electrical measurements to monitor the tissue, the device may also comprise an electrode or series of electrodes to measure electrical properties of the treatment site and provide feedback to the device. Feedback monitoring of current flow is used to modulate the oscillatory speed or extent of travel of the abrasive element. These electrical properties include, but are not limited to, electrical impedence, electrical capacitance or electrical conductivity. Once a desired measure of the electrical property is reached during treatment, feedback to the device may be used to control and monitor further treatment.

Alternatively, feedback control of electrical conductivity of the treated tissue is achieved by using applied magnetic fields and monitoring the eddy currents in the target tissue. By exposing the treated tissue to a magnetic field, for example, at a frequency between 1 kHz and 2.4 GHz, which may be produced by an induction coil or other source that generates an alternating magnetic field, eddy currents are generated in the tissue. Eddy currents induced by a changing magnetic field concentrate near the surface adjacent to an excitation coil. The depth of penetration decreases with increasing frequency and is a function, at least in part, of electrical conductivity in the specimen. Sensitivity to defects and changes in conductivity depends on eddy current density at the target location which may be remotely detected.

Detection of these eddy currents and changes arising as a result of perturbations in the tissues can give useful information on status of the tissue, particularly with regard to local changes in conductivity or hydration (2-3). For example, when stratum corneum is removed from the skin surface, a hydrated layer of skin is revealed that has different conductivity than that of intact skin.

In general, the electrical impedence of the skin can approach values as high as 10⁸ ohms·cm². As successive layers of the stratum corneum are removed, this impedence can drop to a fraction of that value. This drop in impedence can be monitored as a measure of the degree of the process. Another aspect of the invention is that, with the other parameters set, the depth of treatment can be precisely controlled by continuously monitoring the impedence across the target area, and causing a feedback loop whereby the process is halted when a desired endpoint is met. Therefore, various settings on the device can be adjusted to allow successive reduction of the stratum corneum.

Control may be mediated through the creation of a galvanic cell between two monitoring electrodes and fluids encountered in the membrane as a result of treatment. The two monitoring electrodes in electrical contact with the treatment site and untreated site are composed of dissimilar metals. The tip of one electrode is placed adjacent the ablation site on tissue and the electrically conducting dissimilar metal plate of the other electrode is placed in contact with tissue at a location remote from the ablation site.

These electrodes and an electrolyte defined as body fluid present in the intervening tissue below the surface of the skin create a galvanic cell when the tip and plate have different work functions because of migration of electrical charges therebetween. That is when alteration or ablation at the treatment site occurs, charges generated by an electrochemical gradient between the electrodes begin to migrate. This migration of charges is increasingly efficient as the hydration level increases. Thus, the functionality of the galvanic cell may be monitored as a means to detect changes in hydration, and the information used to regulate the energy output of the device. For example, as the successive layers of stratum corneum are removed, the probes encounter a hydration gradient which results in increased conductance.

This last method may optionally require the probe to be in contact with the skin. Alternatively, contact with a liquid interface at the skin surface would minimize the effect of contaminants in the area that may have an electrically insulative effect. The information on conductance is then relayed to a controller which in turn adjusts the treatment of the target site to achieve a desired alteration or ablation. Alternatively, the control means consists of a means to measure the change in the charge storage characteristics of the skin, such that increasing “leakiness” to ions and/or charge, due to breakdown of the “skin battery” is an indication of the depth of treatment.

The device may also monitor endogenous impulses arising from the body by physiological processes, for example, electrical impulses generated by heart. Such impulses may include, but are not limited to, electrical impulses generated by heartbeats. The magnitude of these impulses increases with decreasing electrical resistance of the tissue being treated and so is a measure of the depth of treatment.

Optical properties of the tissue also may be monitored as a means of feedback control. When tissue is altered or ablated, it's optical properties change due either to molecular changes in the tissue itself or due to the exposure of underlying tissue with a different chemical makeup. For example, when the stratum corneum is removed from skin, the underlying epidermis fluoresceses strongly when exposed to the ultraviolet light of a Wood's lamp. When soft tissue is coagulated, it's scattering and absorption properties change and thus the reflectance changes also.

For use of optical measurements to monitor the tissue during alteration or ablation, the control means comprises at least one source of radiant energy, the output of which is directed at the tissue to be interrogated, a light detector with optics such that the interrogated tissue is imaged onto the detector and a controller and microprocessor to modulate the radiant energy source, monitor the detector and to analyze the measurements. The controller further has a microprocessor.

When tissue is altered or ablated, the thermal properties of the tissue change due to molecular alterations in the tissue or due to exposure of underlying tissue of different properties. The properties of thermal diffusivity and thermal conductivity can be monitored by performing pulsed photothermal radiometry whereupon the tissue to be interrogated is heated slightly. For example, the radiant energy of an infrared diode laser directed at the tissue may be used.

The maximum temperature reached and the rate at which the tissue cools are a function of the thermal properties. The temperature of the tissue can be followed by measuring the infrared emission of the tissue with an infrared detector which is optically configured to image the tissue to be interrogated. In the case of skin, when the stratum corneum is completely ablated, a significant change in the infrared emissions from the skin occurs. This abrupt change can be used to controllably ablate the stratum corneum to a reproducible depth.

Depending on the tissue type, when tissue is altered or ablated, transepithelial water loss increases, if the tissue is skin or endothelial tissue, ultrasonic reflection due to changes in acoustic impedance occurs, and respiratory gases, i.e., oxygen and carbon-dioxide, diffuse out. Transepithelial water loss increases when the stratum corneum is altered or ablated because the barrier function of he stratum corneum is compromised and thus diffusion of molecules through the membrane is enhanced; this also explains enhanced gaseous efflux when skin is altered or ablated. Changes in tissue acoustic properties can occur upon alteration or ablation due, in part, to changes in the hydration of the tissue.

Measuring the change in the degree of hydration at the target site can control the depth of treatment, i.e., degree of hydration positively correlates with depth of treatment. Alternatively, by measuring the hydration level in a membrane before the application of a substance, the degree of hydration indicates the likely permeability of a substance through the treated site. The degree of hydration may be determined by corneometry or, preferably, by evaluation of conductance which becomes more efficient as increasing hydration is encountered. Further, the device can seek a pre-determined state of hydration, using this as a benchmark for standardizing permeability of a substance.

A feedback loop is created by a central controller which monitors information on hydration and uses an algorithm to compute relative or absolute hydration. The controller then signals the device to continue or cease the treatment process in order to seek the optimal depth of treatment with respect to hydration and permeability characteristics of a particular substance. The devices described herein are preferably used for alteration or ablation of a membrane, usually the stratum corneum of the skin. The device alters the stratum corneum in a manner that exposes increasingly hydrated layers of this skin layer, thereby increasing the percutaneous absorption of a substance through this layer. When an optimal threshold of hydration is reached the energy delivery is reduced or curtailed.

A desired effect can be obtained by varying the displacement of the piezoelectric actuator and the movement in more than one dimension. Broad surface area treatments may be obtained by applying the device to a treatment surface of greater surface area. The device is moved over the target site on the skin of the individual in order to obtain a large treated surface area. This allows for an efficacious drug dose to be delivered, but avoids local toxic effects due to too high a local concentration of the drug. A representative area of the target site is about 0.1 cm² to about 500 cm².

Additionally, the actuator may be moved in a second or third dimension through the addition of a second actuator, or other driving force, that provides movement in those dimensions. Multiple piezoelectric transducers, or piezoelectric transducers in conjunction with other types of transducers, e.g. electric motors, can be arranged in such a way that motion along multiple axes can occur thus increasing the area of tissue that can be treated and enhancing the lubrication effect which serves to minimize deleterious heat production.

In general, the driving means may be a piezoelectric material, a solenoid, a pressurized gas or an explosive discharge, an electrolytic polymer, a magnetorheological material, an electrorheological material, e.g. dielectric gel mixed with ERF between two flexible electrodes or lithium polymethacrylate, an electroresponsive metal, a shape-memory alloy, or a mechanical spring.

Grit or particle size of the abrasive material is a determining factor in the coarseness of the abrasion with greater particle size relating to greater ablation effect. An abrasive material with particles in the range of about 30 to about 120 microns is preferred, however larger or smaller particles are useful in some applications. The abrasive material may be diamond, aluminum oxide, carborundum which is preferably fixed to a pad driven by the drive mechanism or other material.

Particle size may be chosen to achieve a desired effect. For example, smaller particles may be used for polishing tissue or resurfacing skin for cosmetic or therapeutic purposes as in dermabrasion. Larger abrasive particles may be used to remove tissue. For example a moderate grit or particle of about 30 microns to about 90 microns is used in order to achieve a significant ablation effect while not tearing the tissue or abrading it in an irregular manner. Preferably, the device utilizes an abrasive material of approximately 50 to 90 microns that is driven by a piezoelectric actuator with displacement in the range of 50 to 500 microns. The action of the abrasive is to remove stratum corneum from the skin in order to improve the permeation of substances, such as drugs, into the skin, and the collection of fluids from the body.

Further to the embodiment, particle sizes greater than 120 microns, or smaller than 30 microns, are useful for removal of large amounts of tissue (e.g. bulk debridement) or microsurgical treatment, respectively. Particle sizes below 30 microns are useful where thin layers of tissue are to be removed.

Additionally, the abrasive material may be a pharmaceutical, such as lidocaine, in powdered or frozen crystallized form which acts as the abrasive as well as the drug that subsequently permeates the tissue. In powdered form the drug dissolves upon contact with moisture in the tissue; in the crystallized form the drug melts into a liquid form as a consequence of body heat.

Lubrication of the abrasive member may result from the presence of fluid in the sample, however the lubricating effect of fluid may diminish the abrasive effect of the particles. Nevertheless, an advantage can be derived in that application of a drug may take place simultaneously with the abrasion. Alternatively, a lubricant to irrigate and remove debris, as well as to provide a cooling effect, may be used. This irrigant may be applied as a cryogenic spray to provide an enhanced cooling effect.

Higher frequencies of oscillatory motion result in a more controllable lubricating effect. When particle size is controlled within the given parameters, high frequency treatment results in an effectual uniform surface for treatment, and prevents uncontrolled ablation. Frequencies greater than 1 kHz provide an adequate lubrication effect, however frequencies above approximately 20 kHz can begin to generate too much local heat, potentially resulting in a tissue welding effect. Yet, frequencies above 20 KHz may be desirable as these frequencies are beyond the audible range of the human ear.

A carefully balanced and controlled process using the aforementioned parameters results in successive removal of thin layers of tissue, while balancing heat generation and noise. The layers removed are approximately 1 to 5 micron in thickness. Thus, by controlling pulse number and duration, one may carefully control the depth of treatment at the micron level.

By combining transducers with different operating parameters, it would be possible to produce high-frequencies for adequate lubrication, and provide oscillatory motion. As it is often more difficult to exert great force at higher frequencies, this multi-dimensional approach provides an means in which a high force, relatively low frequency device can be coupled with a high frequency driver, such that the conditions of force and lubrication are achieved. Further, greater displacement in another dimension, which can also be achieved by sacrificing force, allows for relatively large areas of tissue to be treated.

Other parameters which are controllable include the angle of incidence of the actuator with respect to the tissue. By increasing the angle, the ablation effect begins to extend into the membrane in a manner similar to an ultrasonic knife. For micromainpulation of tissue, it is desirable to probe with a fine-tipped actuator coated with the appropriate size abrasive particles. The longitudinal motion of the abrasive members, operating at a high frequency, thus become a lubricated saw.

High frequency vibration minimizes the pressure that must be applied to the surface, thus improving the control over the treatment as well as enabling the use of compact, lightweight applicators that can easily be affixed to the skin surface. In the case of surgical cutting, much less pressure need be applied, thereby minimizing the possibility of distortion of critical membranes or other structures. This “pressureless” surgical tool can provide clean, fast incisions with little or no undesirable damage to surrounding tissues.

One of the limitations of transcutaneous delivery of drug formulations is that the drug can be locally toxic at high doses, and therefore must be modulated to permeate the skin at a controlled rate. In the present case, modulation may occur by limiting the depth of the treatment, and by controlling the flux of the drug by delivering it over a larger surface area. Thus, a large surface area for the delivery of pharmaceutically active substances where those substances may adversely interact with tissues is provided for treatment. Further, substances that have poor permeability characteristics, even in the presence of an altered or ablated membrane, may be better delivered through a larger surface area.

The present invention provides a means for treating local pain or infections, or for application of a substance to a small specified area, directly, thus eliminating the need to provide high, potentially toxic amounts systemically through oral or intravenous administration. Locally acting pharmaceuticals such as alprostadil (for example, Caverject from Pharmacia & Upjohn), various antibiotics, antiviral or antifungal agents, or chemotherapy or anti-cancer agents, can be delivered using this method to treat regions proximal to the delivery site. Protein or DNA based biopharmaceutical agents can also be delivered using this method.

Antigens derived from a virus, bacteria or other agent which stimulates an immune response can be administered through the skin for immunization purposes. The antigen is delivered through the outer layers of the stratum corneum, either singly or multiply, and the immunogen is provided in an appropriate formulation. For booster immunizations, where delivery over a period of time increases the immune response, the immunogen can be provided in a formulation that penetrates slowly through the treatment site, but at a rate faster than possible through unaltered skin.

Analgesics and other non-steroidal anti-inflammatory agents, as well as steroidal anti-inflammatory agents, may be caused to permeate through reduced stratum corneum to locally affect tissue within proximity of the irradiated site. For example, anti-inflammatory agents such as Indocin (Merck & Co.), a non-steroidal drug, are effective agents for treatment of rheumatoid arthritis when taken orally, yet sometimes debilitating gastrointestinal effects can occur. By administering such agents through laser-assisted perforation or alteration sites, these potentially dangerous gastrointestinal complications may be avoided. Further, high local concentrations of the agents may be achieved more readily near the site of irradiation as opposed to the systemic concentrations achieved when orally administered.

The substances used may be a biologic or biological molecules, which may be therapeutics or diagnostics, such as pharmaceutical compounds. Representative examples of such substances are nitroglycerin, an anti-nauseant, a hormone, a steroidal antinflammatory agent, a non-steroid antiinflammatory agent, LHRH, a chemotherapeutic agent, an anti-cancer agent, an immunogen, an anti-viral agent or an anti-fungal agent. A representative example of an anti-nauseant is scopolamine. Representative examples of an antiobiotic are tetracycline, streptomycin, sulfa drugs, kanamycin, neomycin, penicillin, or chloramphenicol. Representative examples of a hormone is parathyroid hormone, growth hormone, gonadotropins, insulin, ACTH, somatostatin, prolactin, placental lactogen, melanocyte stimulating hormone, thyrotropin, parathyroid hormone, calcitonin, enkephalin, or angiotensin. Additionally, the substances of the present invention may be interstitial fluid or a diagnostic reagent. These substances may be removed from tissue using the methods disclosed herein.

The devices provided herein can be used to alter the stratum corneum to improve the collection of fluids, gases or other biomolecules through the skin. The fluid, gas or other biomolecule can be used for a wide variety of tests. A representative example of a use for interstitial fluid is to measure analytes. For example, the technique of the present invention may be used to improve the ability to sample extracellular fluid in order to quantify glucose or other analytes. Glucose is present in the extracellular fluid in the same concentration as or in a known proportion to the glucose level in blood.

The technique of successive removal of layers of dead or necrotic cells of the stratum corneum provides several advantages. Preferably, the stratum corneum is reduced, but not removed, so that its structural and biochemical makeup still permit drugs to permeate. Therefore, the skin after treatment still presents a barrier, albeit reduced, to external factors such as viruses and chemical toxins. Less energy is required for reduction than is required to entirely remove the stratum corneum, thus smaller and cheaper devices can be used. The technique also minimizes the damage to surrounding tissues providing a more rapid and efficient replacement of the stratum corneum.

As described herein, the invention provides a number of therapeutic and diagnostic advantages and uses. Embodiments of the present invention are better illustrated with reference to the Figure(s), however, such reference is not meant to limit the present invention in any fashion. The embodiments and variations described in detail herein are to be interpreted by the appended claims and equivalents thereof.

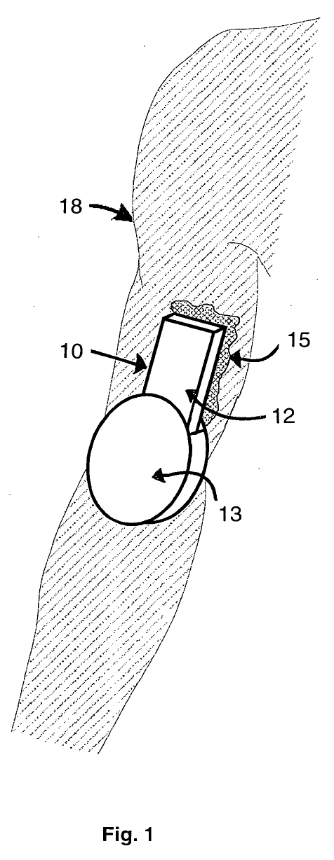

FIG. 1 depicts a device 10 which functions as a vibrating probe having a piezoelectric actuator 12 integrated into a housing 13. The actuator 12 delivers energy resulting in high frequency vibration which causes an ablation or alteration of the membrane 18. An abrasive 15 is applied between the actuator 12 and the membrane 18. Oscillatory movement of the actuator 12 in the plane defined by the membrane 18 causes ablation due to the repeated interaction of the abrasive 15 with the membrane 18.

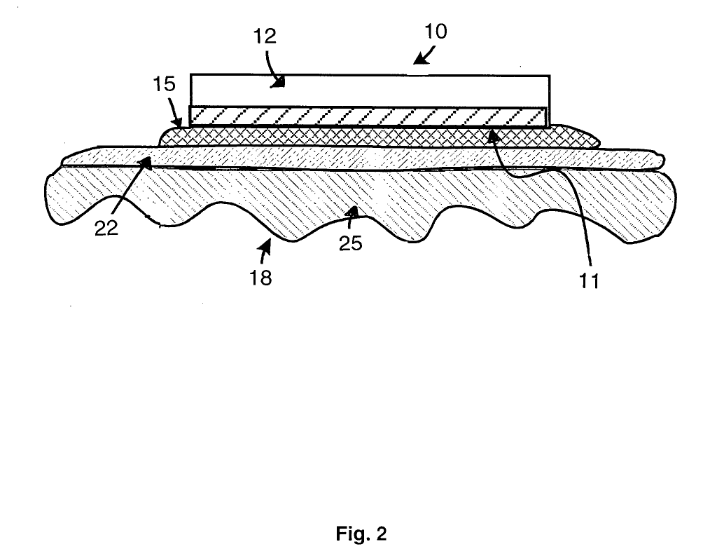

With continued reference to FIG. 1, FIG. 2 depicts a cross-sectional view of the device 10 when used on skin as the membrane 18. Here, the stratum corneum 22 is ablated by the abrasive 15 on the inferior surface 11 of the actuator 12, which is caused to rub back-and-forth due to the high frequency vibratory motion of the actuator 12. For the purpose of enhancing transdermal drug delivery, the depth of ablation does not extend any deeper than the epidermis 25.

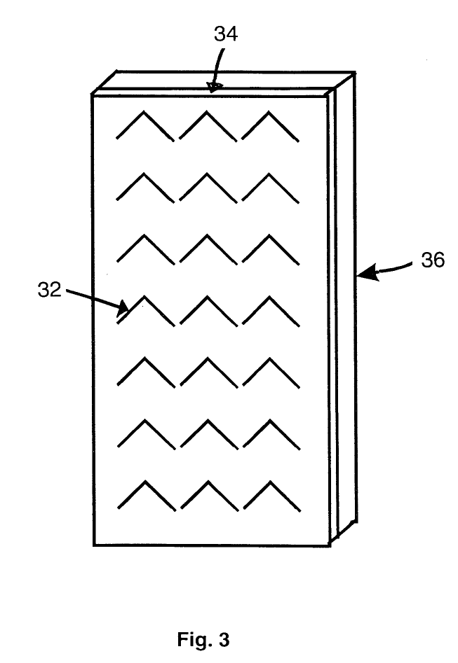

FIG. 3 depicts an embodiment of a modified actuator 36 that is placed against the membrane (not shown) to be treated. An array of chevrons 32 or ridges are disposed on the inferior surface 34 of the modified actuator 36 and extend beyond the inferior surface 34 of the modified actuator 36. With each stroke of the modified actuator 36, ablation takes place and the chevron structures 32 move the ablated material (not shown) to the side of the area being treated. The purpose of this is to remove material that does not take part in the ablative process. Alternatively, the textured actuator surface 34 itself can do the ablation without the need of applying an abrasive, such as shown in FIG. 1, between the actuator 36 and the membrane to be treated (not shown). Other examples of textured actuator surfaces are possible, e.g., random structures such as on sandpaper or microneedles.

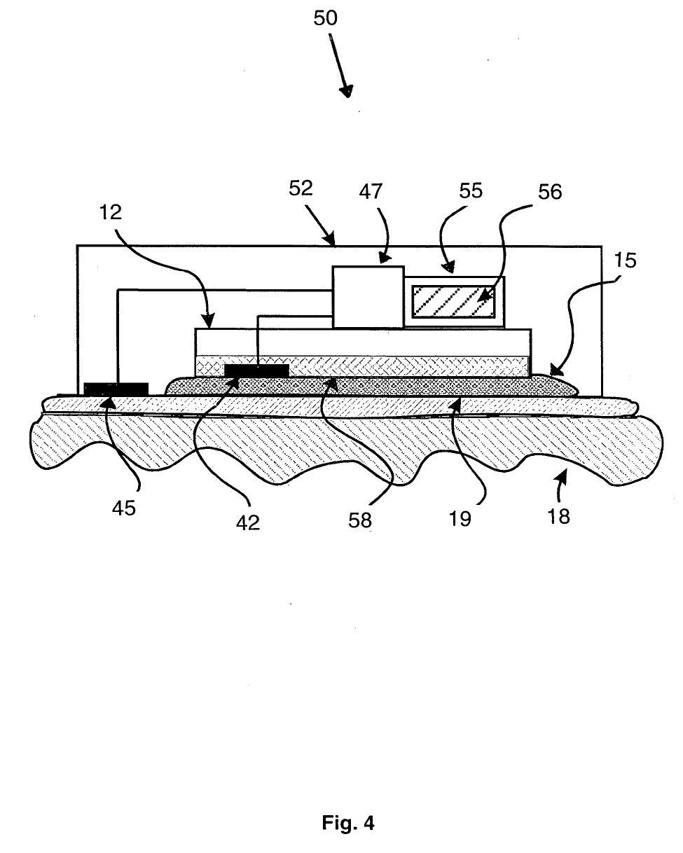

FIG. 4 depicts an alternate embodiment of the device 50. A piezoelectric actuator 12 functioning as a vibrating probe is associated with at least one electrode 42 that is in electrical contact with the ablation site 19 of the membrane 18. An abrasive 15 may be applied on the surface of the membrane 18. Optionally, the abrasive 15 may be fluidized such that a fluid interface is formed that improves the flow of charges between the surface of the electrode 42 and the ablation site 19. A second electrode 45 may be located distally from the first electrode 42 such that the membrane 18 forms a bridge between the electrodes 42, 45 which may be composed of similar or different materials.

A microprocessor (not shown) present in a controller 47 generates a current across the electrodes 42, 45. The controller 47 detects changes in the condition of the treatment site and, according to an algorithm, sends a signal to continue or to cease the delivery of energy until a certain predetermined condition of the treatment site 20 is reached. A patch 52 containing the system contains a substance 56 held in a reservoir 55 to be delivered to the target site 20. In one form of the device a permeable membrane 58 modulates the release of the substance 56 to the treated site 20.

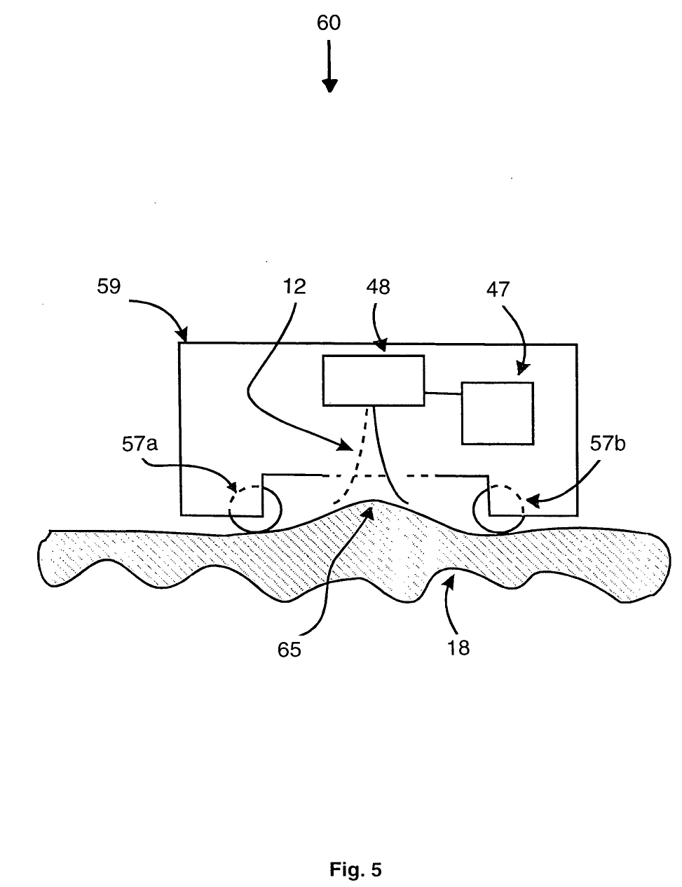

FIG. 5 depicts an alternate embodiment of the device 60. A piezoelectric actuator 12 is configured to vibrate in a bending mode by a power supply 48 and controller 47. The actuator and control electronics are contained within a housing 59 to which is attached cylindrical wheels 57a,b which allow the housing to be moved over the surface of the membrane 18. When downward pressure is applied to the housing 59, the membrane 18 extends upwards into the housing 59 at a position 65 whereupon the vibrating actuator 10 can come into contact with the membrane 18 and cause ablation.

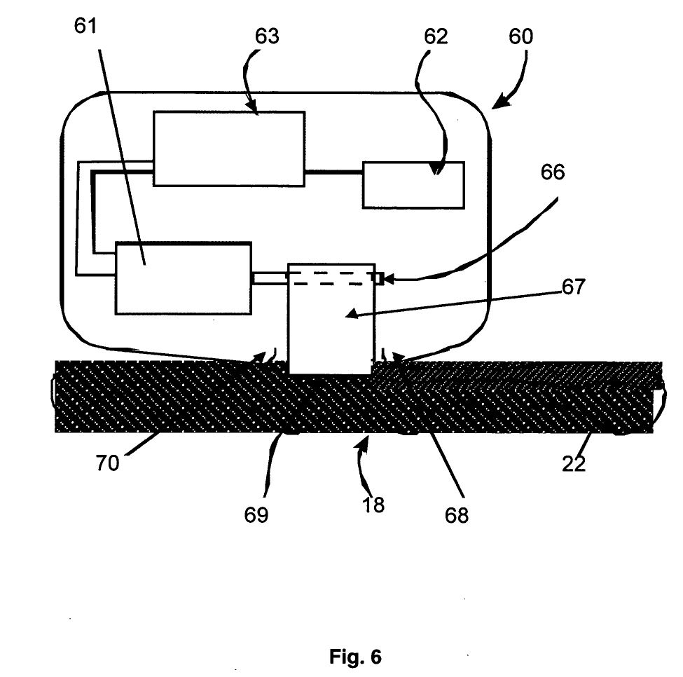

FIG. 6 depicts an alternate embodiment of the device 60 for treating tissue. In this embodiment, an electrical motor 61, controlled and powered by control electronics 62 and a power source 63, is attached to a transducer 67. Attached to the axle 66 of the motor 61 is a asymmetric weight which, when it rotates, either temporarily contacts the tissue 18 with a surface 69, which either ablates or renders the tissue surface, e.g. stratum corneum 22 in skin, ablated or it's barrier function temporarily compromised. The device 60 may optionally have components 68, 70, proximal to the transducer 67, which serve to mechanically make the topography of the tissue surface take on a geometric form most suitable for the transducer. The components 68 and 70 serve to make the device “user friendly” and so not requiring a skilled or experienced operator.

An abrasive may or may not be placed in proximity to the surface 69 of the transducer 67. A pharmaceutical, or other molecule to be administered, may optionally be positioned on the tissue at the surface 69 of the transducer 67 so that permeation may take place during treatment. This may be most beneficial when the device 60 is used as a source of vibrations (subsonic to 20,000 Hz) which, when interacting with tissue, serve to render the barrier function of the tissue temporarily compromised thus enhancing the flux of biomolecules into or out of the tissue.

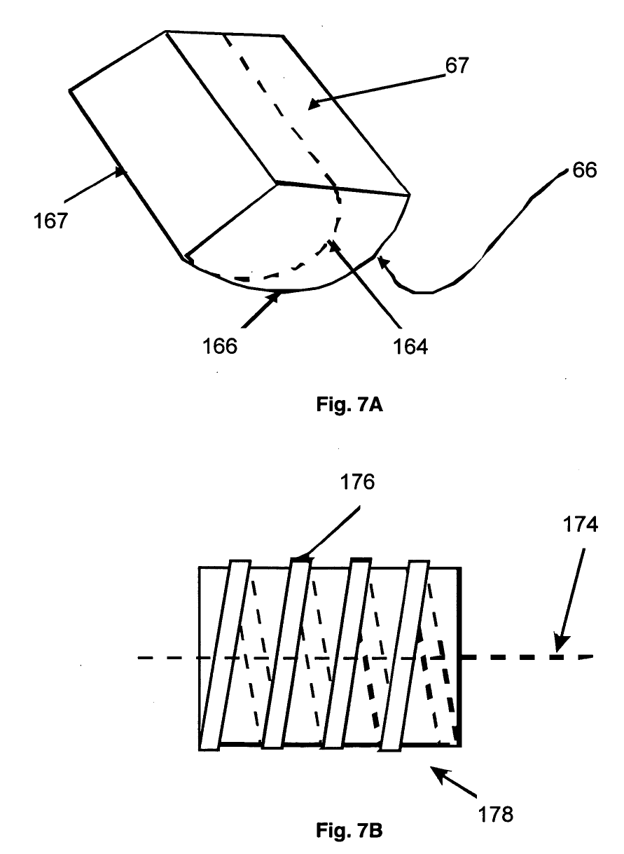

FIG. 7A shows different forms of the transducer 67. The shape of the transducer may be assymetric so that when it rotates, it creates vibrational forces in the device 60 and thus the tissue with which it is in contact. Optionally, the transducer 67 may take the form depicted by the dotted line 164, so that contact with the tissue takes place starting at the edge 167, which may or may not cause cutting, and subsequently contact becomes disabled as the transducer 67 rotates. FIG. 7B, shows an example of a transducer 178, made up of a spiral 176 with sharp edges which, when rotated about the axis 174, causes ablation of the tissue with which it is in contact.

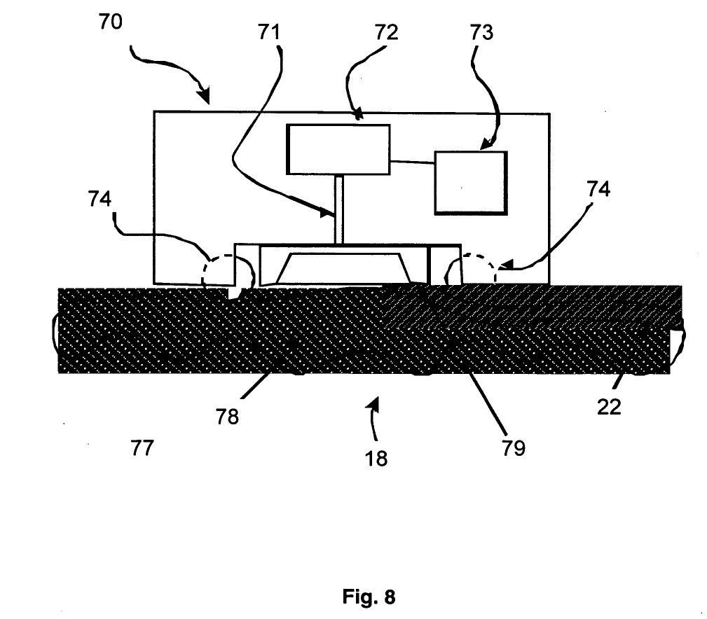

FIG. 8 shows a different form of a device 70 suitable for altering or ablating tissue. In this case, a controller and power supply 73 control a motor 72 which is coupled by an axle 71 to a circular cutting edge 79. The wheels 74, or static structure with a similar function, serve to maintain the topography of the skin plane and parallel to the cutting edge 79.

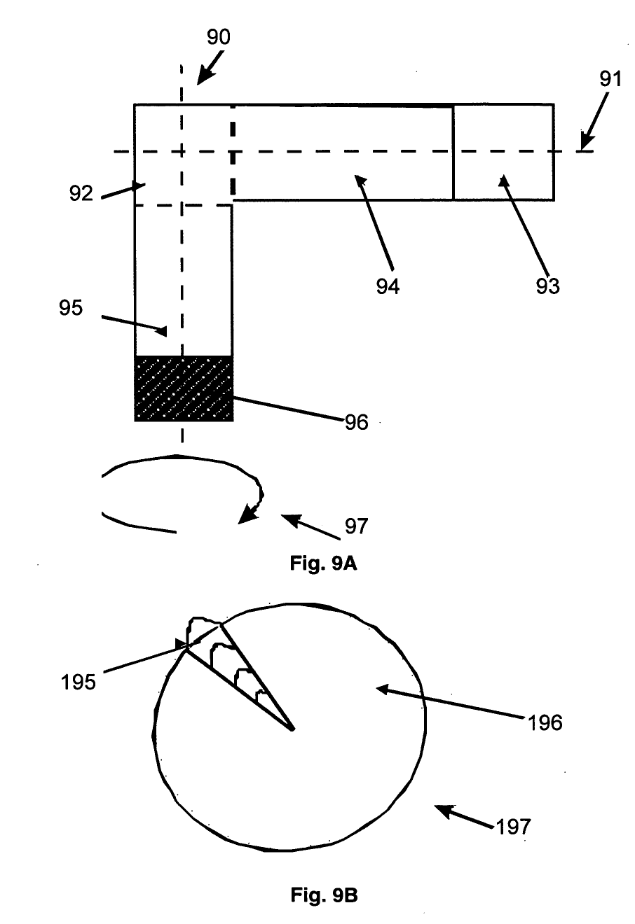

FIGS. 9A-9B shows two different embodiments of transducers suitable for treating tissue. FIG. 9A shows a piezoelectric element 94, fixed at one end 93, and attached at 92 to a second piezoelectric element, one end 96 of which is free to move in a plane defined by the elements 94 and 95. This arrangement serves to produce a motion described by 97, whose motion can vary from a line parallel to axis 90 or 91, to an ellipse or circle. The elements 94 and 95 can be run at different frequencies and/or amplitudes to get the most beneficial tissue effect. If desirable, a piezoelectric element can also be configured to get motion in a dimension perpendicular to the plane defined by element 94 and 95. Exciting the transducer 95 alternately in “stretching mode” and “bending mode”, which can be done by alternating the polarity of the electrode and position of the ground in the electrodes that make up the transducer, can provide motion in multiple dimensions which may be beneficial.

FIG. 9B shows an example of a circular piezoelectric element 197, with radial lines 196 providing a non-uniform texture, and which can be electrically excited to produce a mechanical disruption, in the approximate form of a wave 195, which propagates around the element 197. This transducer configuration can be positioned in contact with tissue, with or without abrasive or pharmaceutical present between the tissue and element 197, and electrically excited to provide the beneficial ablative or altering effect.

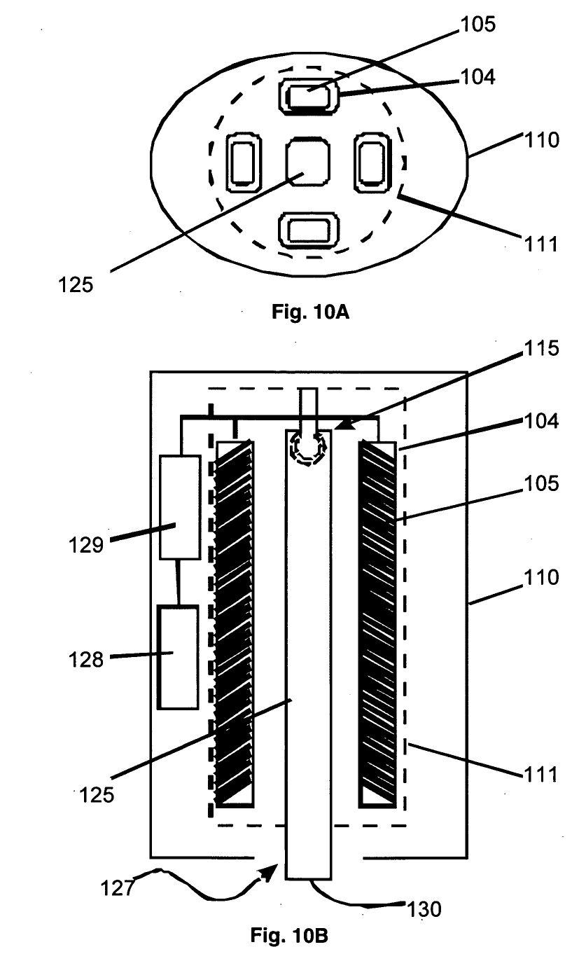

FIGS. 10A and 10B show another device treat tissue. Within a housing 111 are multiple electrical elements 105 consisting of a coil 104 of wire, connected to a source of power 129 and control electronics 128, are positioned around a transducer 125. A housing 110 ergonomcally shaped to be comfortably held in a hand, incorporates the components. The transducer 125 is suspended from the housing 111 with a connector 115, which allows movement in any direction in an axis perpendical to the long axis of the transducer. The actuator 125 has a surface 130, which may or may not have an abrasive texture, and which extends through an opening 127 in the housing 110. Electrical excitation of the elements 105, in a multiplex fashion, results in the formation of a magnetic field which attracts the transducer 125 towards the excited element. In this way, it is possible to produce controllable and rapid motion of the transducer and a beneficial alteration to the surface of the tissue in contract with the surface 130.

The following examples are given to illustrate various embodiments of the invention and are not meant to limit the present invention in any fashion.

### EXAMPLE 1

Ablation of Stratum Corneum Using Micron Size Particles

Optimal particle size for removal of thin tissue layers was determined using a rotational surface applicator device with carborundum or aluminum oxide particles applied to the skin at various rotational frequencies. One adult male volunteer applied pressure at various rotational frequencies in order to determine the efficiency of removing the stratum corneum with regard to frequency and particle size. Frequency of the grinding device varied between 100 and 1000 Hz.

Fixed particle sizes below about 30 microns were inefficient, producing instead only minor abrasion or polishing. When particle size was increased to around 60-74 microns, the stratum corneum was removed efficiently and in a controlled manner as evidenced microscopically. Particle sizes of 100 micron or more produced excessive ablation and were difficult to control. Loose particles of aluminum oxide were found to be less efficient at removal of the outer layer of skin, and larger particle sizes were required, i.e., greater than 120 microns. Significant discomfort was generated during the less than 3 second treatments, possibly as a result of the lack of lubrication due to relatively low speed of the device, resulting in the generation of heat.

### EXAMPLE 2

Ablation of Stratum Corneum Using a Piezoelectric Actuator

The effect of improved lubrication in the treatment area was studied through the use of a high frequency device which was anticipated to reduce the generation of heat. In this study, a piezoelectric actuator was used to apply a reciprocating force in a single direction in the plane of the skin surface, using carborundum or aluminum oxide particles, in an attempt to remove the outer skin layer. Multiple volunteers applied between 0.1 and 4 pounds of force to the area. The displacement of the actuator was between 50 and 250 microns, with a frequency set at 20 kHz. Efficiency of removing the stratum corneum with regard to frequency and particle size was evaluated.

In these experiments, fixed particle sizes below about 30 microns were inefficient, producing minor abrasion, or polishing. When particle size was increased to around 60-74 microns, stratum corneum was removed efficiently and in a controlled manner as evidenced microscopically. Particle sizes of 100 micron or more produced excessive shearing and were difficult to control. Loose particles of aluminum oxide were found to be less efficient at removal of the outer layer of skin, and larger particle sizes were required, i.e., greater than 120 microns. Each pulse of approximately 0.1 to 0.3 seconds removed a thin layer of the stratum corneum as evidenced microscopically and by the generation of a fine dust. Multiple pulses, i.e., between 5 and 20, resulted in complete removal of the stratum corneum and some of the epidermal layer. In some cases, bleeding was observed, however no pain or discomfort was noted.

### EXAMPLE 3

Dermal Resurfacing Using Micron Size Particles

A rotational surface applicator device or planar piezoelectric actuator was used to apply an ablative force parallel to the skin surface using carborundum or aluminum oxide particles to remove the outer skin layer. Several volunteers applied force while testing various frequencies in order to determine the efficiency of polishing the skin with regard to frequency and particle size. Frequency of oscillatory motion of the devices was varied between 100 and 20,000 Hz. In these experiments, fixed particle sizes below about 30 microns were effective in producing minor abrasion, or polishing. Larger particle sizes, up to an exceeding 100 microns, produced significant dermabrasion as well.

### EXAMPLE 4

Delivery of a Topical Anesthetic Through Micro-Ablated Stratum Corneum

A piezoelectric actuator was fitted with 60 micron aluminum oxide particles fixed to its surface so as to provide an approximate 1 cm² treatment area. Ten to fifteen pulses of less than one second duration were applied with force of less than 1 pound to the area and frequency of 20 kHz. Significant ablation was documented by the appearance of fine white powder and redness or edema. A solution of 4% lidocaine was applied to the area and incubated for five minutes. The excess lidocaine was wiped off, and a series of probes was made in and around the area of treatment using a 20 gauge needle or by pinching. In these studies, it was determined that significant anesthesia was obtained through the treatment as evidenced by the lack of sensation within and in close proximity to the treatment site.

### EXAMPLE 5

Microdissection of Tissue

The efficiency of cutting tissue at high frequencies using a piezoelectric actuator fitted with carborundum or aluminum oxide particles where the longitudinal displacement of the actuator was held at a variety of angles relative to the surface to be cut was examined. The displacement of the actuator was between 50 and 250 microns, with a frequency set at 20 kHz. Forces of from 1 to 5 pounds were applied to excised, depilliated sheep skin. Efficiency of removing tissue with regard to frequency and particle size was evaluated. In these studies, each pulse of approximately 0.1 to 0.3 seconds cut into the tissue. When additional pressure was applied, and the angle increased, cutting of the tissue was possible.

The following references are cited herein.

1. Henry et al., Microfabricated Microneedles: A Novel Approach to Transdermal Drug Delivery, Journal of Pharmaceutical Sciences, 8: 922-925 (August 1998).

2. Scharfetter et al., IEEE Transactions on Biomedical Eng. 50(7):870-80 (2003).

3. Scharfetter et al., Physiological Measurement 22(1):131-46 (2001).

Any patents or publications mentioned in this specification are indicative of the levels of those skilled in the art to which the invention pertains. These patents and publications are herein incorporated by reference to the same extent as if it was indicated that each publication was incorporated specifically and individually by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A device for treating a tissue in an individual, comprising:
an applicator having a surface disposed to contact the tissue; and
a means to drive said applicator.

2. The device of claim 1, wherein said means to drive said applicator is single stage actuator or a multi-stage actuator.

3. The device of claim 1, wherein said means to drive said applicator is a piezoelectric actuator, an electrorestrictive actuator, a magnetorestrictive actuator, or a high frequency electronic motor or a combination thereof.

4. The device of claim 1, wherein said means to drive said applicator is magnetically responsive.

5. The device of claim 1, wherein said applicator produces vibratory motion at a frequency from about 1 Hz to about 40,000 Hz.

6. The device of claim 1, wherein said applicator surface comprises an abrasive material.

7. The device of claim 6, wherein said abrasive material has a particle size of about 30 microns to about 120 microns.

8. The device of claim 6, wherein said abrasive is diamond, aluminum oxide or carborundum.

9. The device of claim 6, where said abrasive is crystalline.

10. The device of claim 9, where said abrasive is ice.

11. The device of claim 6, wherein said abrasive comprises a pharmaceutical, a biologic or a diagnostic.

12. The device of claim 1, wherein said applicator surface further comprises a lubricant.

13. The device of claim 12, wherein said lubricant is water, a hydrogel, a lipid, aqueous carbohydrate, petrolatum, an inorganic oil, glycerol or a combination thereof.

13. The device of claim 1, wherein said applicator surface is textured.

14. The device of claim 1, further comprising a means to dispense a therapeutic or diagnostic material.

15. The device of claim 14, wherein said dispensing means comprises:
a reservoir containing said therapeutic or diagnostic material, and
a permeable membrane through which said therapeutic or diagnostic material is controllably released.

16. The device of claim 14, wherein said dispensing means is said abrasive, said abrasive comprising said therapeutic or diagnostic material.

17. The device of claim 16, further comprising a lubricant containing said therapeutic or diagnostic material.

18. The device of claim 17, wherein said therapeutic or diagnostic material is a pharmaceutical.

19. The device of claim 18, wherein said pharmaceutical is an anesthetic, nitroglycerin, an anti-nauseant, an antibiotic, a hormone, a steroidal antinflammatory agent, a non-steroid anti-inflammatory agent, a chemotherapeutic agent, an anti-cancer agent, an immunogen, an anti-viral agent or an anti-fungal agent, a moisturizer, water, or a diagnostic material.

20. The device of claim 19, wherein said antibiotic is tetracycline, streptomycin, sulfa drugs, kanamycin, neomycin, penicillin, or chloramphenicol.

21. The device of claim 19, wherein said hormone is parathyroid hormone, growth hormone, gonadotropins, insulin, ACTH, somatostatin, prolactin, placental lactogen, melanocyte stimulating hormone, thyrotropin, parathyroid hormone, calcitonin, enkephalin, or angiotensin.

22. The device of claim 19, wherein said anesthetic is lidocaine, bupivocaine, tetracaine, morphine, or fentanyl.

23. The device of claim 19, wherein said immunogen is a vaccine.

24. The device of claim 1, further comprising a means to collect ablated tissue or a biomolecule after treating said tissue.

25. The device of claim 24, wherein said collection means is a container operably connected to said device or is an absorptive medium.

26. The device of claim 25, wherein said absorptive medium is activated carbon, a dehydrated hydrogel or cotton.

27. The device of claim 1, further comprising a means to house said device.

28. The device of claim 27, further comprising two wheels rotatably attached to said housing means.

29. The device of claim 1, further comprising a means to monitor an electrical property of the tissue.

30. The device of claim 29, wherein said monitoring means comprises:
at least one active electrode to electrically contact said tissue at a site of interest;
a second return electrode distal to said first electrode to electrically contact said tissue at the site of interest;
an optional electrically conductive fluid interface between said first and second electrodes and the site of interest on said tissue; and
a controller to monitor an electrical current between said first electrode(s) and said second electrode, said controller further comprising a microprocessor.

32. The device of claim 30, wherein said monitoring means comprises:
a source of an alternating magnetic field to generate eddy currents in said tissue;
a detector to detect said eddy currents;
a controller to monitor said eddy currents, said controller further comprising a microprocessor.

33. The device of claim 30, wherein said electrical property is conductivity, impedance or hydration.

34. A method for treating tissue, comprising:
contacting the tissue at a site of interest with the surface of the applicator of the device of claim 1;
actuating said applicator comprising said device via the driving means; and
altering said tissue at the site of interest thereby treating the tissue.

35. The method of claim 34, further comprising:
dispensing a pharmaceutical, a biologic or a diagnostic to said altered tissue.

36. The method of claim 35, wherein said pharmaceutical, biologic or diagnostic is dispensed simultaneously with said tissue alteration or subsequently to said tissue alteration.

37. The method of claim 35, wherein said pharmaceutical is an anesthetic, nitroglycerin, an anti-nauseant, an antibiotic, a hormone, a steroidal antinflammatory agent, a non-steroid antiinflammatory agent, a chemotherapeutic agent, an anti-cancer agent, an immunogen, an anti-viral agent or an anti-fungal agent, a moisturizer, water, or a diagnostic material.

38. The method of claim 37, wherein said antibiotic is tetracycline, streptomycin, sulfa drugs, kanamycin, neomycin, penicillin, or chloramphenicol.

39. The method of claim 37, wherein said hormone is parathyroid hormone, growth hormone, gonadotropins, insulin, ACTH, somatostatin, prolactin, placental lactogen, melanocyte stimulating hormone, thyrotropin, parathyroid hormone, calcitonin, enkephalin, or angiotensin.

40. The method of claim 37, wherein said anesthetic is lidocaine, bupivocaine, tetracaine, morphine, or fentanyl.

41. The method of claim 37, wherein said immunogen is a vaccine.

42. The method of claim 34, further comprising:
monitoring an electrical property of said tissue to control the alteration thereof.

43. The method of claim 42, wherein said monitoring comprises:
contacting said tissue at the site of interest with at least one active electrode and a return electrode distal to said electrode;
monitoring an electrical current between said active electrode(s) and said return electrode via a controller; and
correlating said electric current with the electrical property of said tissue.

44. The method of claim 43, further comprising:
applying an electrically conductive fluid interface between said active electrode(s) and said return electrode and the site of interest on said tissue.

45. The method of claim 43, further comprising:
signaling said device to continue altering said tissue or to adjust the altering of said tissue at the site of interest via a microprocessor in said controller.

46. The method of claim 42, wherein said electrical property is conductivity, impedance or hydration.

47. The method of claim 42, wherein said monitoring comprises:
providing an alternating magnetic field proximally to said tissue;
generating eddy currents via said magnetic field in said tissue;
monitoring said eddy currents via a controller; and
correlating said eddy currents with electrical conductivity of said tissue.

48. The method of claim 47, further comprising:
signaling said device to continue altering said tissue or to adjust the altering of said tissue at the site of interest via a microprocessor in said controller.

49. The method of claim 34, wherein said tissue is membranous or non-membranous.

50. The method of claim 49, wherein said membranous tissue is the stratum corneum.

51. The method of claim 49, wherein said non-membranous tissue is bone.

52. The method of claim 34, wherein treating said tissue comprises at least partial ablation of said tissue, a reduction of the mechanical integrity of said tissue, dermabrasion, or a cosmetic procedure.

53. A method of controlling the permeability of a tissue in an individual comprising:
contacting the tissue at a site of interest with the surface of the applicator of the device of claim 1;
altering said tissue at the site of interest;
monitoring an electrical property of said tissue at the site of interest;
applying an algorithm to evaluate the electrical property;
comparing the value obtained for said electrical property to a predetermined value wherein said values correlate to the permeability of said tissue;
determining if said obtained value is at least equal to said predetermined value; and
signaling the device via the controller to continue altering said tissue if the obtained value does not at least equal the predetermined value thereby controlling the permeability of said tissue at the site of interest.

54. The method of claim 53, wherein said monitoring comprises:
contacting said tissue at the site of interest with at least one active electrode and a return electrode distal to said electrode; and
monitoring an electrical current between said active electrode(s) and said return electrode via a controller.

55. The method of claim 54, further comprising:
applying an electrically conductive fluid interface between said active electrode(s) and and said return electrode and the site of interest on said tissue.

56. The method of claim 53, wherein said monitoring comprises:
providing an alternating magnetic field proximally to said tissue;
generating eddy currents in said tissue via said magnetic field; and
monitoring said eddy currents via a controller.

57. The method of claim 53, further comprising:
dispensing a pharmaceutical to the site of interest, wherein said pharmaceutical is dispensed during said monitoring step or subsequent to reaching said predetermined value of the electrical property.

58. The method of claim 53, wherein said predetermined value of said electrical property is a known value or is obtained prior to treating said tissue.

59. The method of claim 58, wherein said predetermined value of said electrical property is obtained from the same individual or within a group of individuals.

60. The method of claim 43, wherein said electrical property is conductivity, impedance or hydration.
